(19) United States
(12) Patent Application Publication
(10) Pub. No.: US 2022/0339347 A1
(43) Pub. Date: Oct. 27, 2022

(54) SYSTEMS AND METHODS FOR POST-OCCLUSION BOLUS REDUCTION

(71) Applicant: Smiths Medical ASD, Inc., Plymouth, MN (US)

(72) Inventors: Sameer Pai, Plymouth, MN (US); Paul Harrison Coons, Sunfish Lake, MN (US); Quinn Morrison, Minneapolis, MN (US); Grant Adams, Anoka, MN (US)

(21) Appl. No.: 17/638,459

(22) PCT Filed: Aug. 27, 2020
(86) PCT No.: PCT/US2020/070468

§ 371 Date: Feb. 25, 2022

Related U.S. Application Data
(60) Provisional application No. 62/892,707, filed on Aug. 28, 2019.

(51) Int. Cl. A61M 5/142; A61M 5/168

(52) U.S. Cl. CPC A61M 5/14228; A61M 5/16854; A61M 2205/18; A61M 2005/16863

(57) ABSTRACT

A method of adjusting a pressure distribution within an administration set to minimize an inadvertent delivery of a large bolus of infusate upon a sudden release of the occlusion, while ensuring that a maximum safe pressure limitation of the administration set is not exceeded.

### CROSS-REFERENCE TO RELATED APPLICATIONS

The present application claims priority to U.S. Provisional Application No. 62/892,707, filed on Aug. 28, 2019, which is hereby fully incorporated herein by reference.

### TECHNICAL FIELD

The present disclosure relates generally to infusion pump systems, and more particularly, to systems and methods for post-occlusion bolus reduction in large-volume pumps (LVPs) and infusion pump administration sets.

### BACKGROUND

Various types of infusion pumps have been useful for managing the delivery and dispensation of a prescribed amount or dose of a drug, fluid, fluid-like substance, or medicament (herein, collectively, an “infusate”) to patients. Infusion pumps provide significant advantages over manual administration by accurately delivering infusates over an extended period of time. Infusion pumps are particularly useful for treating diseases and disorders that require regular pharmacological intervention, including cancer, diabetes, and vascular, neurological, and metabolic disorders. Infusion pumps also enhance the ability of healthcare providers to deliver anesthesia and manage pain. Infusion pumps are used in various settings, including hospitals, nursing homes, and other short-term and long-term medical facilities, as well as in residential care settings. There are many types of infusion pumps, including ambulatory, large-volume, patient controlled anesthesia (PCA), elastomeric, syringe, enteral, and insulin pumps. Infusion pumps can be used to administer medication through various delivery methods, including intravenously, intraperitoneally, intra-arterially, intradermally, subcutaneously, in close proximity to nerves, and into an inter-operative site, epidural space, or subarachnoid space.

In a particular type of infusion pump system that is commonly referred to as a “peristaltic” pump system, delivery of an infusate to a patient is typically accomplished with the use of an infusion administration set, that is typically disposed of after use and can provide a fluidic pathway (e.g., tubing) for the infusate from a reservoir (such as an intravenous or “IV” bag) to a patient, in cooperation with the pump that controls the rate of flow of the infusate. Peristaltic infusion pumps typically incorporate a peristaltic pumping mechanism that can function by repetitively and temporarily occluding successive sections of tubing of the administration set in a wave-like motion.

A “large-volume pump” or “LVP” system is a common peristaltic pump with related components as aforedescribed. In some publications, the term “volumetric pump” may also be variously used to refer to a peristaltic pump or large-volume pump. While various LVPs have been used in medical environments for years, these devices and their associated peristaltic drive components may have limitations to their efficient, effective and safe usage. In particular, although it is common to have some provision to detect an unwanted occlusion to a prescribed flow of infusate out of the LVP, such as caused by kinked or otherwise unintentionally blocked tubing, such provisions often have the unintended effect of inadvertently delivering a large bolus of infusate upon release of the occlusion and/or a buildup of fluid pressure upstream of the pumping components in excess of the maximum safe tubing/component pressure limitations.

The present disclosure addresses these concerns.

### SUMMARY OF THE DISCLOSURE

Embodiments of the present disclosure provide systems and methods for post-occlusion bolus reduction in large-volume pumps (LVPs) and infusion pump administration sets. In some embodiments, such a system and method provides for strategically adjusting a pressure distribution within an administration set to minimize an inadvertent delivery of a large bolus of infusate upon a sudden release of the occlusion, while ensuring that a maximum safe pressure limitation of the administration set is not exceeded. In doing so, embodiments of the present disclosure consider both a pressure of fluid within the administration set as measured by a downstream pressure sensor, so that removal of the occlusion can be detected, and a pressure of the fluid within the administration set as measured by an upstream pressure sensor to ensure that a maximum safe tubing/component pressure limitation is not exceeded. In some embodiments, adjustment of the pressure distribution is executed via reverse operation of a peristaltic drive mechanism. Once it is detected that the occlusion has been released, for example via a relatively sudden drop in the downstream pressure, normal operations can be automatically restarted to resume infusate delivery.

In an example, the present disclosure provides an infusion pump comprising a pumping mechanism configured to deliver medicament through an infusion set to a patient, a downstream pressure sensor arranged between the pumping mechanism and an outlet of downstream tubing connected to the infusion set, the outlet configured to be coupleable to the patient, an upstream pressure sensor arranged between the pumping mechanism and a source of the medicament connected to the infusion set, and a control unit coupled to the pumping mechanism, the downstream pressure sensor and the upstream pressure sensor. The control unit may be configured to operate the pumping mechanism in a first direction to deliver medicament through the infusion set to the patient, stop the pumping mechanism operating in the first direction in response to an indication from the downstream pressure sensor of a downstream pressure exceeding a first predetermined limit, operate the pumping mechanism in a second direction, the second direction being opposite the first direction, stop the pumping mechanism operating in the second direction in response to an event selected from a group comprising: an indication from the downstream pressure sensor that the downstream pressure has returned to a safe level, and an indication from the upstream pressure sensor of an upstream pressure reaching or exceeding a second predetermined limit, and automatically restart operation of the pumping mechanism in the first direction in response to an indication from the downstream pressure sensor that the downstream pressure has returned to the safe level.

In an example, the present disclosure provides a method of operating an infusion pump to prevent post-occlusion bolus, the infusion pump including a pumping mechanism, a downstream pressure sensor and an upstream pressure sensor. The method may be performed by the infusion pump and comprises operating the pumping mechanism in a first direction to deliver medicament to a patient, monitoring downstream pressure with the downstream pressure sensor, stopping the pumping mechanism operating in the first direction in response to an indication from the downstream pressure sensor of a downstream pressure exceeding a first predetermined limit, operating the pumping mechanism in a second direction to reduce possibility of inadvertent delivery of a bolus to the patient, the second direction being opposite the first direction, monitoring upstream pressure with the upstream pressure sensor, comparing downstream pressure with the downstream pressure sensor to a predetermined safe level, and comparing upstream pressure with the upstream pressure sensor to a second predetermined limit, stopping the pumping mechanism in the second direction in response to an event selected from a group comprising: an indication from the downstream pressure sensor that the downstream pressure has returned to the safe level, and an indication from the upstream pressure sensor of the upstream pressure reaching or exceeding the second predetermined limit, and automatically restarting operation of the pumping mechanism in the first direction in response to an indication from the downstream pressure sensor that the downstream pressure has returned to a safe level.

In an example, the present disclosure provides an infusion pump including a pumping mechanism, a downstream pressure sensor and an upstream pressure sensor. The infusion pump may be configured to operate the pumping mechanism in a first direction to deliver medicament through an infusion set to a patient, stop the pumping mechanism operating in the first direction in response to an indication from the downstream pressure sensor of a downstream pressure exceeding a first predetermined limit, operate the pumping mechanism in a second direction to reduce possibility of inadvertent delivery of a bolus to the patient, the second direction being opposite the first direction, compare downstream pressure with the downstream pressure sensor to a predetermined safe level, and compare upstream pressure with the upstream pressure sensor to a second predetermined limit, stop the pumping mechanism operating in the second direction in response to an event selected from a group comprising: an indication from the downstream pressure sensor that the downstream pressure has returned to the safe level, and an indication from the upstream pressure sensor of an upstream pressure reaching or exceeding the second predetermined limit, and automatically restart operation of the pumping mechanism in the first direction in response to an indication from the downstream pressure sensor that the downstream pressure has returned to the safe level.

The summary above is not intended to describe each illustrated embodiment or every implementation of the present disclosure. The figures and the detailed description that follow more particularly exemplify these embodiments.

### BRIEF DESCRIPTION OF THE DRAWINGS

The disclosure can be more completely understood in consideration of the following detailed description of various embodiments of the disclosure, in connection with the accompanying drawings, in which:

FIG. 1 is a schematic perspective view depicting a peristaltic infusion pump system for use with a patient, in accordance with an embodiment of the disclosure.

FIG. 2A is a schematic perspective view depicting portions of the peristaltic infusion pump of FIG. 1, particularly illustrating an assembly receptacle and receptacle door, in accordance with an embodiment of the disclosure.

FIG. 2B is a schematic perspective view depicting portions of the peristaltic infusion pump of FIG. 2A, with a portion of an administration set received by the assembly receptacle, in accordance with an embodiment of the disclosure.

FIG. 3 is a schematic view depicting various components and electrical circuitry of a peristaltic infusion pump system, in accordance with an embodiment of the disclosure.

FIG. 4 is a flowchart depicting a method for post occlusion bolus reduction, in accordance with an embodiment of the disclosure.

FIG. 5A is a first exemplary graphical representation depicting an upstream and downstream pressure during an execution of the method depicted in FIG. 4, in accordance with an embodiment of the disclosure.

FIG. 5B is a second exemplary graphical representation depicting an upstream and downstream pressure during an execution of the method depicted in FIG. 4, in accordance with an embodiment of the disclosure.

While embodiments of the disclosure are amenable to various modifications and alternative forms, specifics thereof shown by way of example in the drawings will be described in detail. It should be understood, however, that the intention is not to limit the disclosure to the particular embodiments described. On the contrary, the intention is to cover all modifications, equivalents, and alternatives falling within the spirit and scope of the subject matter as defined by the claims.

### DETAILED DESCRIPTION

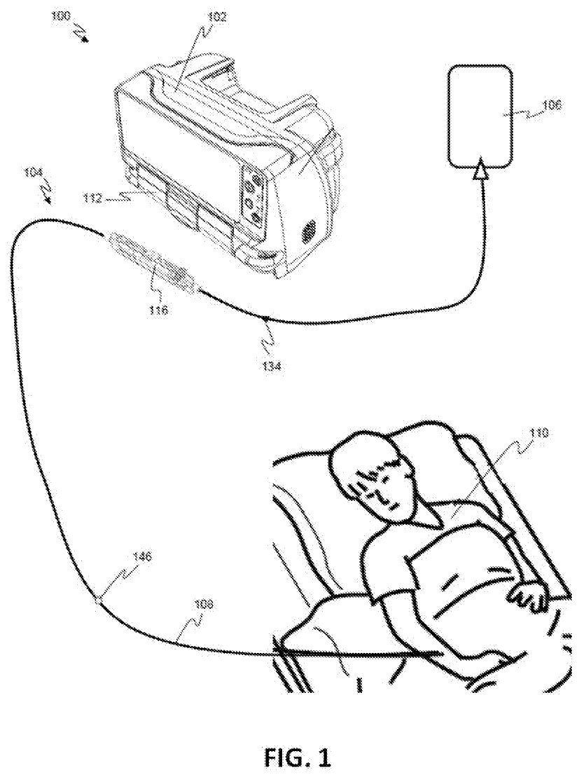

FIG. 1 is a schematic perspective view of an example embodiment of a peristaltic infusion pump system 100 for use with a patient, that includes a peristaltic pump 102 (more specifically, an LVP pump 102) and a disposable administration set 104 that is structured and configured to be operably and removably coupleable to the pump 102. Administration set 104 is schematically shown providing a fluidic pathway from an IV bag 106 to an infusion set or tubing 108 that ultimately delivers infusate(s) to a patient 110. In FIG. 1, the receptacle door 112 of the peristaltic pump 102 is shown in a closed configuration and the administration set 104 is illustrated as not coupled to the pump 102.

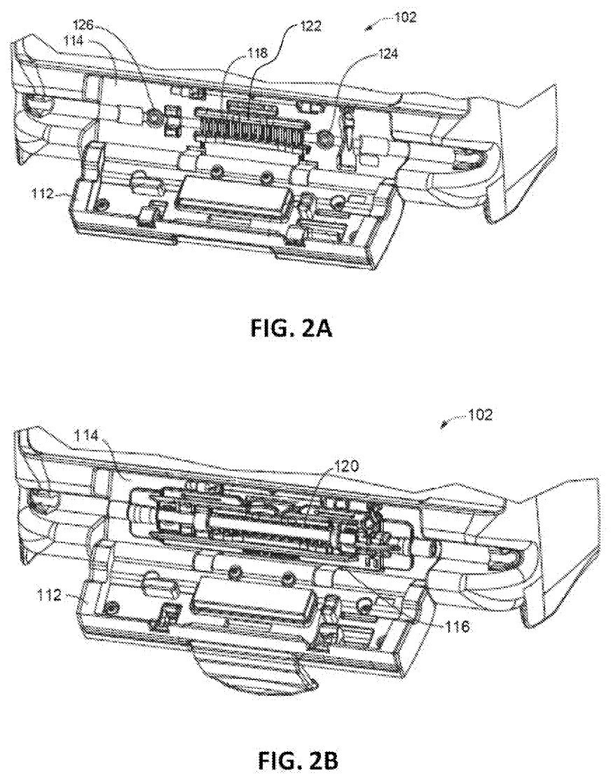

To more fully illustrate various components of the pump 102, FIG. 2A and FIG. 2B show a partial depiction of the pump 102. Specifically, only a portion of the pump 102 in proximity to an assembly receptacle 114 and receptacle door 112 is shown. The assembly receptacle 114 can be configured to receive an assembly 116 of the administration set 104, such that the administration set 104 is thereby operably coupled to the pump 102. In particular, FIG. 2B is a schematic perspective view of portions of the peristaltic infusion pump 102 of FIG. 2A, with assembly 116 received by or installed in the assembly receptacle 114. The receptacle door 112 can be opened or closed to allow or block access to the assembly receptacle 114. In both FIGS. 2A-B, the receptacle door 112 of the pump 102 is in an open position.

A linear peristaltic pump drive mechanism 122 can be located in the assembly receptacle 114. Assembly 116 of the administration set 104 can be configured and structured to position elements of the administration set 104, including a centrally located segment of the tube 120 of the assembly 116 in an operative relationship with the peristaltic drive mechanism 122. The centrally located segment of tube 120 can be formed of a resilient material that is suitable for compression (and recovery from compression) by the peristaltic drive mechanism 122 of the pump 102. The peristaltic pump mechanism 122 can include tube engaging members 118 (sometimes referred to as “fingers”), configured to urge, push, force, or otherwise transport fluid through the administration set 104 by repetitively and temporarily squeezing or occluding the centrally located segment of tube 120 in a wave-like motion.

FIGS. 2A-B depict the pump 102 including twelve tube engaging members 118; in other embodiments, fewer or additional tubing engaging members may be present. In general, the number and/or size of tube engaging members 118 can in part determine the quantity of fluid delivery for each pump cycle or the “packet size” of fluid being delivered. For example, in one embodiment, the packet size of fluid can be 13 μL; other packet sizes are also contemplated.

Fluid pressure generated within the administration set 104 is generally detectable via an elastic stretching or deformation of portions of the administration set 104. For example, in one embodiment, fluid pressures within the administration set 104, upstream and downstream of the tube engaging members 118, is detectable by an upstream pressure sensor 124 and a downstream pressure sensor 126. As depicted in FIGS. 2A-B, the upstream pressure sensor 124 and downstream pressure sensor 126 can be located within the assembly receptacle 114 on each respective side of the tube engaging members 118. Other locations, combinations and arrangements of sensors are also contemplated.

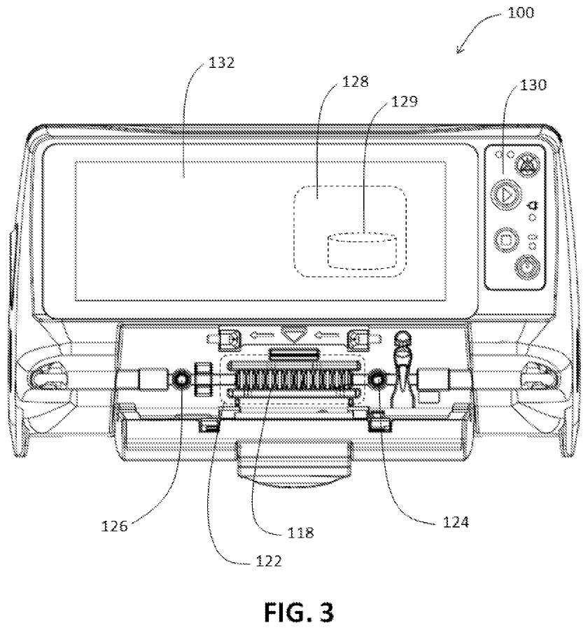

FIG. 3 is a schematic view of various components and electrical circuitry within the infusion pump system 100. The tube engaging members 118 can be driven by the peristaltic drive mechanism 122, which can be controlled by a control unit 128 having a memory 129. The control unit 128 can receive inputs from a keypad 130, and other input devices, sensors and monitors, such as the upstream pressure sensor 124 or the downstream pressure sensor 126. The control unit 128 can also provide an output and receive input from a graphical user interface 132 such as, for example, a touch-screen input and display system.

In one embodiment, the control unit 128 can continually sense a downstream pressure via the downstream pressure sensor 126 to monitor for an occlusion. Should an occlusion be detected, the control unit 128 can direct the peristaltic drive mechanism 122 to operate in reverse to control a pressure buildup of the infusate within the administration set 104 to inhibit the inadvertent delivery of a large bolus of infusate upon release of the occlusion. Simultaneously, the control unit can monitor an upstream pressure via the upstream pressure sensor 124 to ensure that while operating the peristaltic drive mechanism 122 in reverse, the pressure within upstream portions of the administration set 104 remain below a designated maximum safe pressure limit. Once the infusion pump system 100 detects that the occlusion has been released, for example via a relatively sudden drop in the downstream pressure, the infusion pump system 100 can automatically restart normal operations to resume infusate delivery.

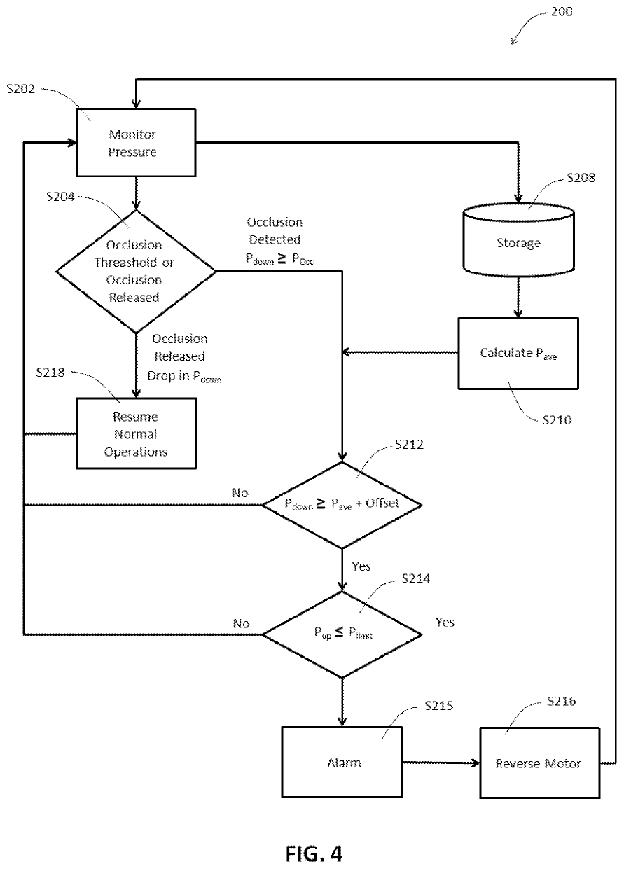

Referring to FIG. 4, a method 200 for post-occlusion bolus reduction is depicted in accordance with an embodiment of the disclosure. At S202, an upstream pressure (Pᵤₚ) and downstream pressure (P_{down}) within the administration set 104 can be monitored, for example via the upstream pressure sensor 122 and the downstream pressure sensor 126. The pressure sensors 124/126 respond to forces exerted by an expansion of the flexible tubing of the administration set 104 in response to increasing fluid pressure therewithin. Data measured by the upstream and downstream pressure sensors 124/126 representing Pᵤₚ and P_{down} can be sent to the control unit 128 for evaluation. The control unit 128 includes memory 129 containing information as to an upper maximum predetermined occlusion pressure limit (P_{occ}).

At S204, P_{down} is compared to P_{occ}. If P_{down} is less than P_{occ}, then no occlusion has been detected and the infusion pump system 100 continues normal infusate delivery. However, if P_{down} is greater than or equal to P_{occ}, then infusion pump system 100 infers that an obstruction has occurred in the administration set 104, which is usually caused by an occlusion in the path of infusate downstream from the peristaltic drive mechanism 122. The downstream pressure sensor 126 thereby operates as an occlusion detector.

If an occlusion is detected, the control unit 128 can stop forward drive by the peristaltic drive mechanism 122, to alleviate or stop further deleterious pressure build-up in administration set 104. Most commonly, such an occlusion is caused by a kink in the infusion line, potentially by the patient unknowingly and temporarily rolling onto or otherwise bending the infusion line in a manner that inhibits a flow of fluid therethrough. Accordingly, a pressure within the administration set 104 upstream of the kink or occlusion will rise as long as the peristaltic drive mechanism 122 continues to operate in the forward direction. Even after an occlusion has been detected, and the drive mechanism 122 has been stopped, the pressure within the administration set 104 between the occlusion and the drive mechanism 122 will remain at a heightened pressure (e.g., the pressure at the time that the drive mechanism 122 stops). A sudden release of the occlusion (e.g., the infusion line suddenly becoming unkinked) could cause the pressurized fluid to be delivered to the patient in a large bolus of infusate, which with some types of infusates can be dangerous.

To reduce the possibility of inadvertently delivering a large bolus of infusate after the sudden release of an occlusion, once an occlusion has been detected, embodiments of the present disclosure can run the peristaltic drive mechanism 122 in reverse for the purpose of reducing the pressure in the administration set 104 between the occlusion and the tube engaging members 118. However, because administration sets 104 frequently include an upstream check valve 134 (as depicted in FIG. 1), to inhibit a backflow of infusate through the administration set 104 and into the IV bag 106, a pressure buildup in an administration set 104 with an occlusion cannot typically be relieved downstream of the check valve 134 until the occlusion has been removed.

Accordingly, embodiments of the present disclosure seek to readjust a pressure distribution within the administration set 104 between the occlusion and the check valve 134. In doing so, embodiments of the present disclosure considers both a pressure of the fluid as measured by the downstream pressure sensor 126, so that removal of the occlusion can be detected, and pressure of the fluid as measured by the upstream pressure sensor 124 to ensure that the maximum safe tubing/component pressure limitations are not exceeded. In some embodiments, this is performed through a two-step decision process as depicted in FIG. 4.

In particular, at S212, P_{down} is compared to a calculated average downstream pressure (Pₐᵥₑ) plus an offset factor, which in one embodiment can be a percentage (e.g., between about 5-10% of the occlusion threshold). Pₐᵥₑ is calculated at S210 based on data from the downstream pressure sensor 126 collected in the memory 129 of control unit 128. If P_{down} is less than Pₐᵥₑ plus the offset, then the method 200 proceeds to S202 to monitor the downstream pressure via the downstream pressure sensor 126. If P_{down} is greater than or equal to Pₐᵥₑ plus the offset, the method 200 proceeds to S214, where the upstream pressure is evaluated.

At S214, the upstream pressure (Pᵤₚ), for example as measured by the upstream pressure sensor 124, is compared to the maximum safe tubing/component pressure limit (Pₗᵢₘᵢₜ). If Pᵤₚ is less than Pₗᵢₘᵢₜ, then the method 200 proceeds to S202 to monitor the upstream and downstream pressure via the pressure sensors 124/126. But if Pᵤₚ is greater than or equal to Pₗᵢₘᵢₜ, then at S215, the control unit 128 can provide an alarm signal, such an audible alarm and/or warning indication on the graphical user interface 132. At S216, the control unit 128 drives the peristaltic drive mechanism 122 in reverse, thereby transferring fluid within the portion of the administration set 104 between the occlusion and the tube engaging members 118 upstream to the portion of the administration set 104 between the tube engaging members 118 and the check valve 134. According to the method 200, this process continues until either: (1) P_{down} reaches Pₐᵥₑ plus an offset factor, or (2) Pᵤₚ reaches Pₗᵢₘᵢₜ.

While the peristaltic drive mechanism 122 is operating in reverse, at S202, the system 100 continues to monitor the downstream pressure via the downstream pressure sensor 126. If a relatively sudden drop in downstream pressure is detected at S204, indicating a release of the occlusion, the alarm can be discontinued and at S218 the control unit 128 can automatically restart normal operations to resume infusate delivery.

It should be understood that the individual steps used in the methods of the present disclosure may be performed in any order and/or simultaneously, as long as the disclosure remains operable. Furthermore, it should be understood that the systems and methods of the present disclosure can include any number, or all, of the described embodiments, as long as the disclosure remains operable.

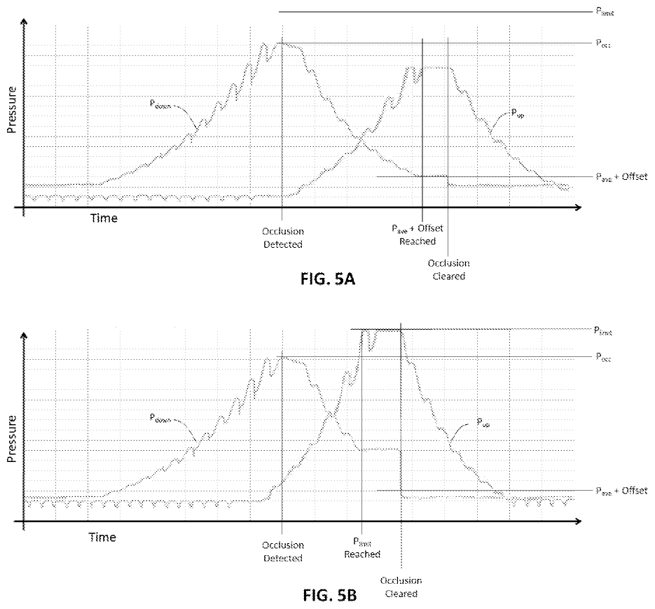

FIG. 5A depicts an exemplary graphical representation of the downstream pressure (P_{down}) and the upstream pressure (Pᵤₚ) over a period of time where an occlusion is detected, the pressure within the administration set 104 is adjusted to inhibit the inadvertent delivery of a large bolus of infusate upon a sudden release of the occlusion while ensuring that the maximum safe pressure limitations of the administration set 104 are not exceeded, followed by a release of the occlusion. As can be seen in this example, Pₗᵢₘᵢₜ is approached but not actually reached by the upstream pressure, thereby permitting operation of the peristaltic drive mechanism 122 in reverse until the downstream pressure reaches Pₐᵥₑ plus the offset. Release of the occlusion is observed when the upstream pressure rather suddenly drops. Thereafter, normal forward operation of the peristaltic drive mechanism 122 is automatically restarted and Pᵤₚ is gradually reduced.

FIG. 5B depicts a second exemplary graphical representation of the downstream pressure (P_{down}) and the upstream pressure (Pᵤₚ) over a period of time, in which Pₗᵢₘᵢₜ is reached and drive of the peristaltic drive mechanism 122 in reverse is halted. Accordingly, in this embodiment, P_{down} is held above Pₐᵥₑ plus the offset, indicating that the magnitude of any inadvertent bolus delivery upon release of the occlusion has been minimized to the extent possible within the maximum safe tubing/component pressure limitations of the administration set 104.

Various embodiments of systems, devices, and methods have been described herein. These embodiments are given only by way of example and are not intended to limit the scope of the claimed subject matter. It should be appreciated, moreover, that the various features of the embodiments that have been described may be combined in various ways to produce numerous additional embodiments. Moreover, while various materials, dimensions, shapes, configurations and locations, etc. have been described for use with disclosed embodiments, others besides those disclosed may be utilized without exceeding the scope of the claimed subject matter.

Persons of ordinary skill in the relevant arts will recognize that the subject matter hereof may comprise fewer features than illustrated in any individual embodiment described above. The embodiments described herein are not meant to be an exhaustive presentation of the ways in which the various features of the subject matter hereof may be combined. Accordingly, the embodiments are not mutually exclusive combinations of features; rather, the various embodiments can comprise a combination of different individual features selected from different individual embodiments, as understood by persons of ordinary skill in the art. Moreover, elements described with respect to one embodiment can be implemented in other embodiments even when not described in such embodiments unless otherwise noted.

Although a dependent claim may refer in the claims to a specific combination with one or more other claims, other embodiments can also include a combination of the dependent claim with the subject matter of each other dependent claim or a combination of one or more features with other dependent or independent claims. Such combinations are proposed herein unless it is stated that a specific combination is not intended.

Any incorporation by reference of documents above is limited such that no subject matter is incorporated that is contrary to the explicit disclosure herein. Any incorporation by reference of documents above is further limited such that no claims included in the documents are incorporated by reference herein. Any incorporation by reference of documents above is yet further limited such that any definitions provided in the documents are not incorporated by reference herein unless expressly included herein.

For purposes of interpreting the claims, it is expressly intended that the provisions of 35 U.S.C. § 112(f) are not to be invoked unless the specific terms “means for” or “step for” are recited in a claim.

What is claimed is:

1. An infusion pump, comprising:
a pumping mechanism configured to deliver medicament through an infusion set to a patient;
a downstream pressure sensor arranged between the pumping mechanism and an outlet of downstream tubing connected to the infusion set, the outlet configured to be coupleable to the patient;
an upstream pressure sensor arranged between the pumping mechanism and a source of the medicament connected to the infusion set; and
a control unit coupled to the pumping mechanism, the downstream pressure sensor and the upstream pressure sensor, the control unit configured to:
operate the pumping mechanism in a first direction to deliver medicament through the infusion set to the patient;
stop the pumping mechanism operating in the first direction in response to an indication from the downstream pressure sensor of a downstream pressure exceeding a first predetermined limit;
operate the pumping mechanism in a second direction, the second direction being opposite the first direction;
stop the pumping mechanism operating in the second direction in response to an event selected from a group comprising: an indication from the downstream pressure sensor that the downstream pressure has returned to a safe level, and an indication from the upstream pressure sensor of an upstream pressure reaching or exceeding a second predetermined limit; and
automatically restart operation of the pumping mechanism in the first direction in response to an indication from the downstream pressure sensor that the downstream pressure has returned to the safe level.

2. The infusion pump of claim 1, wherein the control unit further includes memory containing the first predetermined limit and the second predetermined limit.

3. The infusion pump of claim 1, wherein the upstream pressure sensor is arranged between the pumping mechanism and a check valve arranged on upstream tubing connected to the infusion set.

4. The infusion pump of claim 1, wherein the safe level is based on an average calculated from data received from the downstream pressure sensor.

5. The infusion pump of claim 4, wherein the safe level includes an offset factor.

6. The infusion pump of claim 1, wherein the control system is further configured to provide an alarm signal in response to an indication from the downstream pressure sensor of the downstream pressure exceeding the first predetermined limit.

7. The infusion pump of claim 1, wherein the first predetermined limit is less than the second predetermined limit.

8. A method of operating an infusion pump to prevent post-occlusion bolus, the infusion pump including a pumping mechanism, a downstream pressure sensor and an upstream pressure sensor, the method being performed by the infusion pump and comprising:
operating the pumping mechanism in a first direction to deliver medicament to a patient;
monitoring downstream pressure with the downstream pressure sensor;
stopping the pumping mechanism operating in the first direction in response to an indication from the downstream pressure sensor of a downstream pressure exceeding a first predetermined limit;
operating the pumping mechanism in a second direction to reduce possibility of inadvertent delivery of a bolus to the patient, the second direction being opposite the first direction;
monitoring upstream pressure with the upstream pressure sensor;
comparing downstream pressure with the downstream pressure sensor to a predetermined safe level, and comparing upstream pressure with the upstream pressure sensor to a second predetermined limit;
stopping the pumping mechanism in the second direction in response to an event selected from a group comprising: an indication from the downstream pressure sensor that the downstream pressure has returned to the safe level, and an indication from the upstream pressure sensor of the upstream pressure reaching or exceeding the second predetermined limit; and
automatically restarting operation of the pumping mechanism in the first direction in response to an indication from the downstream pressure sensor that the downstream pressure has returned to a safe level.

9. The method of claim 8, further comprising providing an alarm signal in response to an indication from the downstream pressure sensor of the downstream pressure exceeding the first predetermined limit.

10. The method of claim 8, wherein comparing downstream pressure with the downstream pressure sensor to a predetermined safe level further comprises comparing downstream pressure with the downstream pressure sensor to the predetermined safe level including an offset factor.

11. The method of claim 8, wherein the safe level is based on an average calculated from data received from the downstream pressure sensor during operation of the infusion pump.

12. The method of claim 8, wherein the first predetermined limit is less than the second predetermined limit.

13. An infusion pump including a pumping mechanism, a downstream pressure sensor and an upstream pressure sensor, the infusion pump configured to:
operate the pumping mechanism in a first direction to deliver medicament through an infusion set to a patient;
stop the pumping mechanism operating in the first direction in response to an indication from the downstream pressure sensor of a downstream pressure exceeding a first predetermined limit;
operate the pumping mechanism in a second direction to reduce possibility of inadvertent delivery of a bolus to the patient, the second direction being opposite the first direction;
compare downstream pressure with the downstream pressure sensor to a predetermined safe level, and compare upstream pressure with the upstream pressure sensor to a second predetermined limit;
stop the pumping mechanism operating in the second direction in response to an event selected from a group comprising: an indication from the downstream pressure sensor that the downstream pressure has returned to the safe level, and an indication from the upstream pressure sensor of an upstream pressure reaching or exceeding the second predetermined limit; and
automatically restart operation of the pumping mechanism in the first direction in response to an indication from the downstream pressure sensor that the downstream pressure has returned to the safe level.

14. The infusion pump of claim 13, wherein the safe level includes an offset factor.

15. The infusion pump of claim 14, wherein the safe level is based on an average calculated from data received from the downstream pressure sensor during operation of the infusion pump.
